(19) United States
(12) Patent Application Publication
(10) Pub. No.: US 2010/0075992 A1
(43) Pub. Date: Mar. 25, 2010

(54) Tetrodotoxin and its derivatives for the treament of peripheral-nervously derived neuropathi pain

(75) Inventors: Helmut Buschmann, Barcelona (ES); Frank Hay Kong Shum, North Vancouver (CA); Kim Noel Fisher, Chestermere (CA); Anh Ho Ngoc, Montreal (CA); Michel Hamon, Paris Cedex (FR)

Correspondence Address: FISH & RICHARDSON PC, P.O. BOX 1022, MINNEAPOLIS, MN, 55440-1022, US

(73) Assignee: LABORATORIOS DEL DR. ESTEVE S.A., BARCELONA (ES)

(21) Appl. No.: 11/663,358

(22) PCT Filed: Sep. 20, 2005
(86) PCT No.: PCT/EP05/10136

§ 371 Date: Oct. 9, 2008

Related U.S. Application Data
(60) Provisional application No. 60/611,753, filed on Sep. 22, 2004.

(51) Int. Cl. A61K 31/519; A61P 25/00

(52) U.S. Cl. 514/257

(57) ABSTRACT

The present invention refers to the use of a sodium channel blocker such as tetrodotoxin or saxitoxin, their analogues and derivatives as well as their physiologically acceptable salts, in medicinal products for human therapeutics for the treatment of peripheral-nervously derived neuropathic pain.

### FIELD OF THE INVENTION

The present invention refers to the use of a sodium channel blocker such as tetrodotoxin or saxitoxin, their analogues and derivatives as well as their physiologically acceptable salts, in medicinal products for human therapeutics for the treatment of peripheral-nervously derived neuropathic pain.

### BACKGROUND OF THE INVENTION

The treatment of pain conditions is of great importance in medicine. There is currently a world-wide need for additional pain therapy. The pressing requirement for a specific treatment of pain conditions or as well a treatment of specific pain conditions which is right for the patient, which is to be understood as the successful and satisfactory treatment of pain for the patients, is documented in the large number of scientific works which have recently and over the years appeared in the field of applied analgesics or on basic research on nociception.

PAIN is defined by the International Association for the Study of Pain (IASP) as “an unpleasant sensory and emotional experience associated with actual or potential tissue damage, or described in terms of such damage (IASP, Classification of chronic pain, 2^{nd} Edition, IASP Press (2002), 210). Even though pain is always subjective its causes or syndromes can be classified.

Especially neuropathic pain which in the past years has developed into a major health problem in broad areas of the population needs a very specific treatment, especially considering that any treatment of neuropathic pain is extremely sensitive to the causes behind the pain, be it the disease ultimately causing it or the mechanistic pathway over which it develops. So, in a majority of cases a substance being able to treat one subtype of neuropathic pain is not—or is at least not necessarily—able to treat other specific subtypes due to the highly diverse nature of this generalized symptom called neuropathic pain.

Therefore, it was the underlying problem solved by this invention to find new ways of treating neuropathic pain, in this case peripheral-nervously derived neuropathic pain.

So, the main object of this invention is the use of a sodium channel blocker and/or one of its derivatives for the production of a medicament for the treatment of peripheral-nervously derived neuropathic pain. The sodium channel blocker is optionally used in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate.

It was found out that TTX is acting on peripheral-nervously derived neuropathic pain with a surprising and also extremely high potency.

The term “sodium channel blocker” mentioned in this application is defined as a compound that specifically binds to and specifically inhibits sodium channels, which are classified as either TTX-resistant or TTX-sensitive. The term TTX-resistant” and TTX-sensitive refers to a difference in the tightness of TTX binding, with the TTX resistant channel having a binding constant as mentioned in Hunter et al., Current Opinion in CPNS Investigational Drugs 1 (1), 1999 as well as Clare et al. DDT, 5 (11), 2000, 506-520 included here by reference and the TTX sensitive channel having a binding constant as mentioned in Hunter et al., Current Opinion in CPNS Investigational Drugs 1 (1), 1999 as well as Clare et al. DDT, 5 (11), 2000, 506-520. A preferred sodium channel blocker thus binds to a sodium channel with a IC₅₀ of less than 200 □M, preferably less than 100 □M or with an IC₅₀ of 2 □M. Said inhibition refers to suppression or modification of any downstream effect caused by activation of said sodium channels. More preferably, the term “sodium channel blocker” mentioned in this invention refers to compounds binding to an alpha subunit of sodium channels, especially TTX-resistant or TTX-sensitive sodium channels. More preferably, the term “sodium channel blocker” mentioned in this invention refers to compounds binding to either a SS1 or SS2 region of an alpha subunit of sodium channels, especially TTX-resistant or TTX-sensitive sodium channels. Preferred sodium channel blockers for use in this invention are tetrodotoxin and saxitoxin which both specifically inhibit said sodium channels.

The term “analogues” as used in this application is defined here as meaning a chemical compound that is a derivative of a compound which has similar biochemical activity to that compound. “Analogues” of TTX and STX bind to the same site on the alpha subunit of sodium channels as does TTX and STX.

The term “derivatives” as used in this application is defined here as meaning a chemical compound having undergone a chemical derivation such as substitution or addition of a further chemical group to change (for pharmaceutical use) any of its physico-chemical properties, such as solubility or bioavailability. Derivatives include so-called prodrugs, e.g. ester and ether derivatives of an active compound that yield the active compound per se after administration to a subject.

Examples of well known methods of producing a prodrug of a given acting compound are known to those skilled in the art and can be found e.g. in Krogsgaard-Larsen et al., Textbook of Drugdesign and Discovery, Taylor & Francis (April 2002).

In connection with this invention “neutral form” refers to the non-ionic form but also to (at its isoelectric point) neutrally loaded forms (that means containing an equal amount of positive and negative loads) especially the Zwitter-lon.

The term “salt” according to this invention is to be understood as meaning any form of the active compound according to the invention in which this compound assumes an ionic form (even in solution) or is charged and—if applicable—is also coupled with a counter-ion (a cation or anion). By this are also to be understood complexes of the active compound with other molecules and ions, in particular complexes which are complexed via ionic interactions. As preferred examples of salts this includes the acetate, mono-trifluoracetate, acetate ester salt, citrate, formate, picrate, hydrobromide, monohydrobromide, monohydrochloride or hydrochloride.

The term “physiologically acceptable salt” in the context of this invention is understood as meaning a “salt” (as defined above) of at least one of the compounds according to the invention which are physiologically tolerated—especially if used in humans and/or mammals.

The term “solvate” according to this invention is to be understood as meaning any form of the active compound according to the invention in which this compound has attached to it via non-covalent binding another molecule (most likely a polar solvent) especially including hydrates and alcoholates, e.g. methanolate.

The term “treatment” or “to treat” in the context of this specification means administration of a compound or formulation according to the invention to prevent, ameliorate or eliminate one or more symptoms associated with peripheral-nervously derived neuropathic pain. Furthermore, the terms “to treat” or “treatment” according to this invention include the treatment of symptoms of peripheral-nervously derived neuropathic pain especially certain subtypes of peripheral-nervously derived neuropathic pain, the treatment of the consequences causing the symptoms, the prevention or the prophylaxis of the symptoms of peripheral-nervously derived neuropathic pain, especially certain subtypes of peripheral-nervously derived neuropathic pain.

The term “ameliorate” in the context of this invention is understood as meaning any improvement on the situation of the patient treated—either subjectively (feeling of or on the patient) or objectively (measured parameters).

“Neuropathic pain” is defined by the IASP as “pain initiated or caused by a primary lesion or dysfunction in the nervous system” (IASP, Classification of chronic pain, 2^{nd} Edition, IASP Press (2002), 210). For the purpose of this invention included under this heading or to be treated as synonymous is “Neurogenic Pain” which is defined by the IASP as “pain initiated or caused by a primary lesion, dysfunction or transitory perturbation in the peripheral or central nervous system”. By the restriction of the use according to the current invention to “peripheral-nervously derived” it is clear that the use is restricted to pain caused or initiated in the peripheral nervous system.

The term “peripheral-nervously derived neuropathic pain” according to this invention is to be understood as meaning a neuropathic pain being initiated or caused by a primary lesion, dysfunction or transitory perturbation in the peripheral nervous system, whereas the “peripheral nervous system” is herewith defined as the nervous system without the brain and the spinal cord

In a highly preferred use according to the invention the sodium channel blocker is selected from tetrodotoxin or any of its derivatives or analogues and/or saxitoxin or any of its derivatives or analogues, optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate.

In another highly preferred use according to the invention the sodium channel blocker is selected from tetrodotoxin, optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate.

Tetrodotoxin (alternatively in the context of this application abbreviated TTX), also known as Ti Qu Duo Xin, is an alkaloid found in puffer fish (Tetradontiae). The chemical name is Octahydro-12-(Hydroxymethyl)-2-imino-5,9,7,10a-dimethano-10aH-[1,3]dioxocino[6,5-d]pyrimidine-4,7,10,11,12-pentol with a molecular formula C₁₁H₁₇N₃O₈ and a Molecular weight of 319.27. It is a potent non-protein neurotoxin and an indispensable tool for the study of neurobiology and physiology. Tetrodotoxin (TTX) is a marine organic toxin which is mainly found in testicles, ovaries, eggs, livers, spleens, eyeballs, and blood of puffer fish as well as in diverse animal species, including goby fish, newt, frogs and the blue ringed octopus and even in marine alga. Several processes for producing TTX are known. Usually TTX is extracted from marine organisms (e.g. JP 270719 Goto and Takahashi) but besides numerous others methods of synthesis are also described (and used for the preparation of tetrodotoxin in connection to this invention) in U.S. Pat. No. 6,552,191, U.S. Pat. No. 6,478,966, U.S. Pat. No. 6,562,968 or 2002/0086997, all of which are included here by reference. Tetrodotoxin is a well known compound described for example in WO02/22129 as systemically acting as analgesic. For one of the many descriptions of TTX it is recommended turn to e.g. Tu, Anthony (Ed.) Handbook of Natural Toxins, Vol. 3: Marine Toxins and Venoms, 1988, 185-210 as well as Kao (1966), Pharmacol. Rev. 18:997-1049 and others.

Older journals mention that based on the method described by Tahara in U.S. Pat. No. 1,058,643, there was a product sold in Japan containing a 1% solution of TTX extract for uses such as enuresis (Iwakawa and Kimura, Archiv fuer Experimentelle Pathologie and Pharmakologie (1922), 93, 305-31). There were also trials in the 1930s (Hsiang, Nai Shi; Manshu Igaku Zasshi (1939), 30, 639-47 (German abstr. 179) testing the abilities of TTX for addiction treatment.

Tetrodotoxin is a well known compound described for example in CN 1145225 as acting as an analgesic as well as in the treatment of drug addiction. WO02/22129 describes TTX as systemically acting as an analgesic, including acting on neuropathic pain. This general mentioning of neuropathic pain as an example of pain to be treated with TTX is not dealing with any specific subtype of neuropathic pain, especially not with peripheral-nervously derived neuropathic pain.

The phrase “its (tetrodoxin's) derivatives and analogues” according to this invention is defined—using the definition of U.S. Pat. No. 6,030,974 (included here by reference)—as meaning amino perhydroquinazoline compounds having the molecular formula C₁₁H₁₇N₃O₈. Another definition of “tetrodoxin's derivatives and analogues” according to this invention refers to the definition of U.S. Pat. No. 5,846,975 (included here by reference) as amino hydrogenated quinazolines and derivatives including the substances defined from column 3 line 40 to column 6 line 40. Specifically defined “derivatives and analogues of tetrodotoxin” according to this invention are including but are not limited to anhydro-tetrodotoxin, tetrodaminotoxin, methoxytetrodotoxin, ethoxytetrodotoxin, deoxytetrodotoxin and tetrodonic acid, 6 epi-tetrodotoxin, 11-deoxytetrodotoxin as well as the hemilactal type TTX analogues (e.g. 4-epi-TTX, 6-epi-TTX, 11-deoxy-TTX, 4-epi-11-deoxy-TTX, TTX-8-O-hemisuccinate, chiriquitoxin, 11-nor-TTX-6(S)-ol, 11-nor-TTX-6(R)-ol, 11-nor-TTX-6,6-diol, 11-oxo-TTX and TTX-11-carboxylic acid), the lactone type TTX analogues (e.g. 6-epi-TTX (lactone), 11-deoxy-TTX (lactone), 11-nor-TTX-6(S)-ol (lactone), 11-nor-TTX-6(R)-ol (lactone), 11-nor-TTX-6,6-diol (lactone), 5-deoxy-TTX, 5,11-dideoxy-TTX, 4-epi-5,11-didroxy-TTX, 1-hydroxy-5,11-dideoxy-TTX, 5,6,11-trideoxy-TTX and 4-epi-5,6,11-trideoxy-TTX) and the 4,9-anhydro type TTX analogs (e.g. 4,9-anhydro-TTX, 4,9-anhydro-6-epi-TTX, 4,9-anhydro-11-deoxy-TTX, 4,9-anhydro-TTX-8-O-hemisuccinate, 4,9-anhydro-TTX-1′-O-hemisuccinate). The typical analogues of TTX possess only ⅛ to 1/40 of the toxicity of TTX in mice, based upon bioassay in mice. It has been observed that the analogues produce joint action, and do not interact adversely. Examples of TTX analogues include novel TTX analogs isolated from various organisms, as well as those that are partially or totally chemically synthesized (see e.g., Yotsu, M. et al. Agric. Biol. Chem., 53(3):893-895 (1989)). Such analogues bind to the same site on the alpha subunit of sodium channels as does TTX.

According to U.S. Pat. No. 6,030,974, “saxitoxin” or “STX” refers to a compound comprising a tetrahydropurine moiety composed of two guanidine units fused together in a stable azaketal linkage, having a molecular formula CloHl7N704 (mol. wt. 299.30) and to derivatives thereof, including but not limited to hydroxysaxitoxins and neosaxitoxin. Bower et al, Nonprotein Neurotoxins, Clin. Toxicol. 18 (7): 813-863 (1981).

It is to be understood that the use according to the invention is restricted to peripheral-nervously derived neuropathic pain in regards to all the paintypes mentioned in here.

In a highly preferred embodiment of the use according to the invention the peripheral-nervously derived neuropathic pain is peripheral neuropathic pain or peripheral neurogenic pain.

According to the IASP “peripheral neuropathic pain” is defined as “a pain initiated or caused by a primary lesion or dysfunction in the peripheral nervous system” and “peripheral neurogenic pain” is defined as “a pain initiated or caused by a primary lesion, dysfunction or transitory perturbation in the peripheral nervous system” (IASP, Classification of chronic pain, 2^{nd} Edition, IASP Press (2002), 213).

In another preferred embodiment of the use according to the invention the peripheral-nervously derived neuropathic pain is allodynia.

According to the IASP “allodynia” is defined as “a pain due to a stimulus which does not normally provoke pain” (IASP, Classification of chronic pain, 2^{nd} Edition, IASP Press (2002), 210).

In another preferred embodiment of the use according to the invention the peripheral-nervously derived neuropathic pain is causalgia.

According to the IASP “causalgia” is defined as “a syndrome of sustained burning pain, allodynia and hyperpathia after a traumatic nerve lesion, often combined with vasomotor and sudomotor dysfunction and later trophic changes” (IASP, Classification of chronic pain, 2^{nd} Edition, IASP Press (2002), 210).

In another preferred embodiment of the use according to the invention the peripheral-nervously derived neuropathic pain is hyperalgesia.

According to the IASP “hyperalgesia” is defined as “an increased response to a stimulus which is normally painful(IASP, Classification of chronic pain, 2^{nd} Edition, IASP Press (2002), 211).

In another preferred embodiment of the use according to the invention the peripheral-nervously derived neuropathic pain is hyperesthesia.

According to the IASP “hyperesthesia” is defined as “increased sensitivity to stimulation, excluding the senses” (IASP, Classification of chronic pain, 2^{nd} Edition, IASP Press (2002), 211).

In another preferred embodiment of the use according to the invention the peripheral-nervously derived neuropathic pain is hyperpathia.

According to the IASP “hyperpathia” is defined as “a painful syndrome characterized by an abnormally painful reaction to a stimulus, especially a repetitive stimulus, as well as an increased threshold” (IASP, Classification of chronic pain, 2^{nd} Edition, IASP Press (2002), 212).

The IASP draws the following difference between “allodynia”, “hyperalgesia” and “hyperpathia” (IASP, Classification of chronic pain, 2^{nd} Edition, IASP Press (2002), 212):

| | | |
|---|---|---|
| Allodynia | Lowered threshold | Stimulus and response |
| | | mode differ |
| Hyperalgesia | Increased response | Stimulus and response |
| | | rate are the same |
| Hyperpathia | Raised threshold; | Stimulus and response |
| | Increased response | rate may be the same |
| | | or different |

In another preferred embodiment of the use according to the invention the peripheral-nervously derived neuropathic pain is neuralgia.

According to the IASP “neuralgia” is defined as “Pain in the distribution of a nerve or nerves” (IASP, Classification of chronic pain, 2^{nd} Edition, IASP Press (2002), 212).

In another preferred embodiment of the use according to the invention the peripheral-nervously derived neuropathic pain is neuritis.

According to the IASP “neuritis” is defined as “Inflammation of a nerve or nerves” (IASP, Classification of chronic pain, 2^{nd} Edition, IASP Press (2002), 212).

In another preferred embodiment of the use according to the invention the peripheral-nervously derived neuropathic pain is neuropathy.

According to the IASP “neuritis” is defined as “a disturbance of function or pathological change in a nerve: in one nerve mononeuropathy, in several nerves mononeuropathy multiplex, if diffuse and bilateral, polyneuropathy” (IASP, Classification of chronic pain, 2^{nd} Edition, IASP Press (2002), 212).

In human therapeutics, the dose administered is normally between 10 and 4000 μg/day of the sodium channel blocker, especially tetrodotoxin, its derivatives or its analogues, especially the dose of e.g. tetrodotoxin administered is normally between 10 and 4000 μg/day or—given the likely twice per day treatment—between 5 to 2000 μg each given dose, sometimes preferably between 250 and 1000 μg each given dose, sometimes preferably between 25 and 50 μg each given dose depending on the route of administration.

In connection with this invention any amount defined refers to each compound individually not to any combination and refers to the compound having a purity of ≧97%. This on the other hand will exclude any impurity contained within the >3% to be mentioned, defined or referred to as active compound in the sense of this invention. For example this would mean that a formulation containing 0.5 mg tetrodotoxin of 99% purity and 0.8% anhydro-tetrodotoxin will be classified and defined according to this invention as containing just tetrodotoxin as active ingredient.

In a highly preferred embodiment of the invention the use according to the invention the sodium channel blocker, especially the tetrodotoxin, its derivative and/or one of its analogues is used in an amount between 10 μg/day and 4 mg/day.

In a highly preferred embodiment of the invention the used tetrodotoxin, its derivative or its analogue is isolated from a biological source, preferably from fish, especially puffer fish.

In a highly preferred embodiment of the invention the used tetrodotoxin, its derivative or its analogue is synthesized.

Any formulation or pharmaceutical composition according to the invention contains the active ingredient (e.g a sodium channel blocker like TTX (Tetrodotoxin), its derivatives and/or its analogues) as well as optionally at least one auxiliary material and/or additive and/or optionally another active ingredient.

The auxiliary material and/or additive can be specifically selected from conserving agents, emulsifiers and/or carriers for parenteral application. The selection of these auxiliary materials and/or additives and of the amounts to be used depends upon how the pharmaceutical composition is to be applied. Examples include here especially parenteral like intravenous subcutaneous or intramuscular application formulations but which could also be used for other administration routes. The most preferred route is generally systemical, preferably meaning not for local action. Still topical routes are also possible.

Routes of administration of tetrodotoxin its derivatives and its analogues can include intramuscular injection, intravenous injection, subcutaneous injection, sublingual, bucal, patch through skin, oral ingestion, implantable osmotic pump, collagen implants, aerosols or suppository.

Included in this invention are especially also methods of treatments of a patient or a mammal, including men, suffering from peripheral-nervously derived neuropathic pain using a sodium channel blocker such as tetrodotoxin or saxitoxin and/or one of its analogues or derivatives optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate. It is also preferred if the method of treatment is restricted to tetrodotoxin, optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate. It is also preferred if the method of treatment is restricted to tetrodotoxin, in neutral form or as a salt, especially a physiologically acceptable salt, whereas preferably tetrodotoxin, its derivative and/or one of its analogues is used in an amount between 10 μg/day and 4 mg/day, is isolated from a biological source, preferably from fish, especially puffer fish, or is synthesized.

The examples and figures in the following section describing pharmacological trials are merely illustrative and the invention cannot be considered in any way as being restricted to these applications.

### FIGURES

FIGS. 1 to 4 referring to example 2 are described there.

### EXAMPLES

### Example 1

### Example Formulation of an Injectable (Im/Iv) Solution of TTX

| | | |
|---|---|---|
| Tetrodotoxin (TTX) (powdered material) | 15 | mg |
| 0.5% diluted acetic acid | 1 | ml |
| Acetic Acid - actetate buffer solution (pH = 3-5) | 50 | ml |
| Water for injection c.s.p., add to | 1000 | ml |

The dosage of TTX for injection is 30 μg in 2 ml.

### Example 2

### Neuropathic Pain

### Dose-Effect Study of TTX-Phase I (Bennet Model): Unilateral Ligature of the Sciatic Nerve (Mechanical Response Thresholds)

According to the Model described by Bennet (Bennet and Xie, Pain 1988, 33, 87-107) a well established model for neuropathic pain, rats were operated with the following surgical procedure:

Rats were anaesthesized using Pentobarbital. Then the skin was incised. Following that a sciatic nerve ligature was placed and the incision was closed with silk thread.

In Sham-operated control animals the identical surgical procedures were followed, but the nerve was not ligatured.

In these dose effect study of TTX activity TTX was administered subcutaneously (s.c.) was measured at 15′, 30′, 45′, 1 h, 2 h, 3 h, 4 h, 5 h, 6 h.

The measurement was done to determine the thresholds to paw pressure and von Frey filaments;

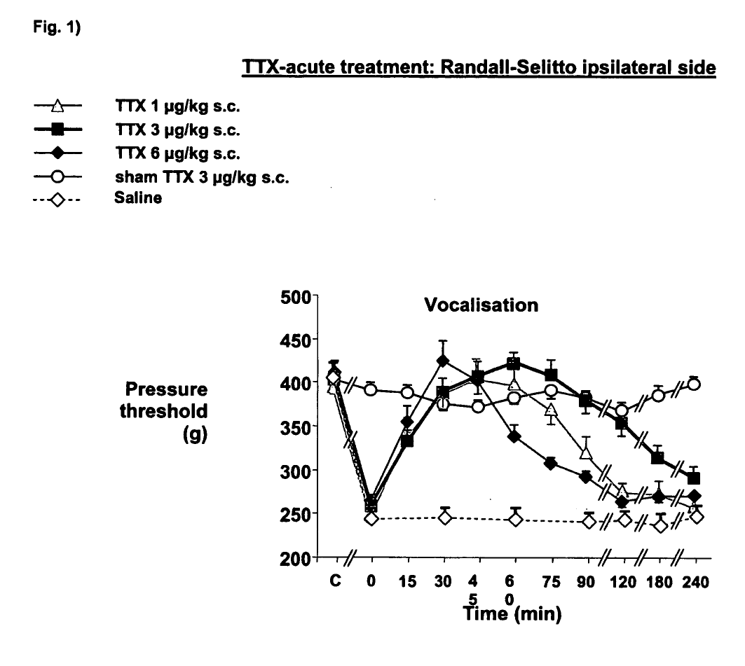

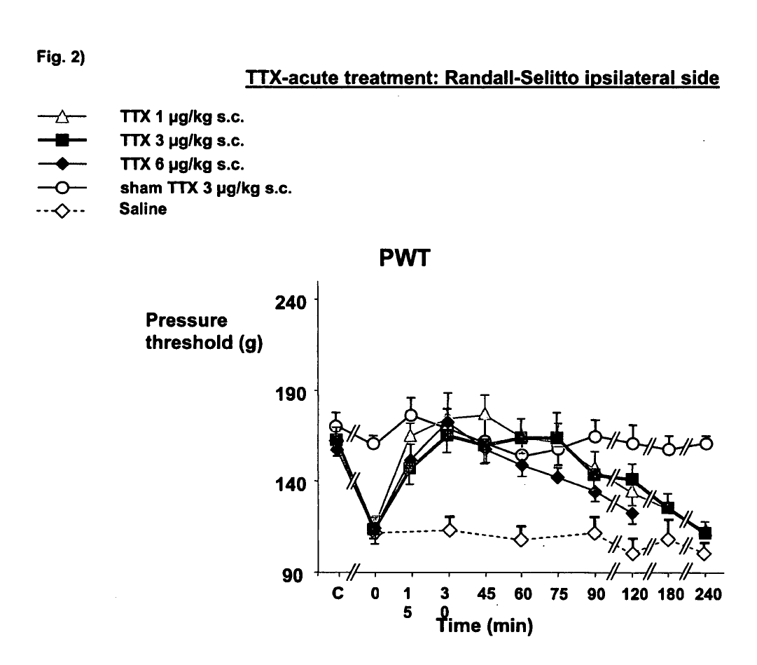

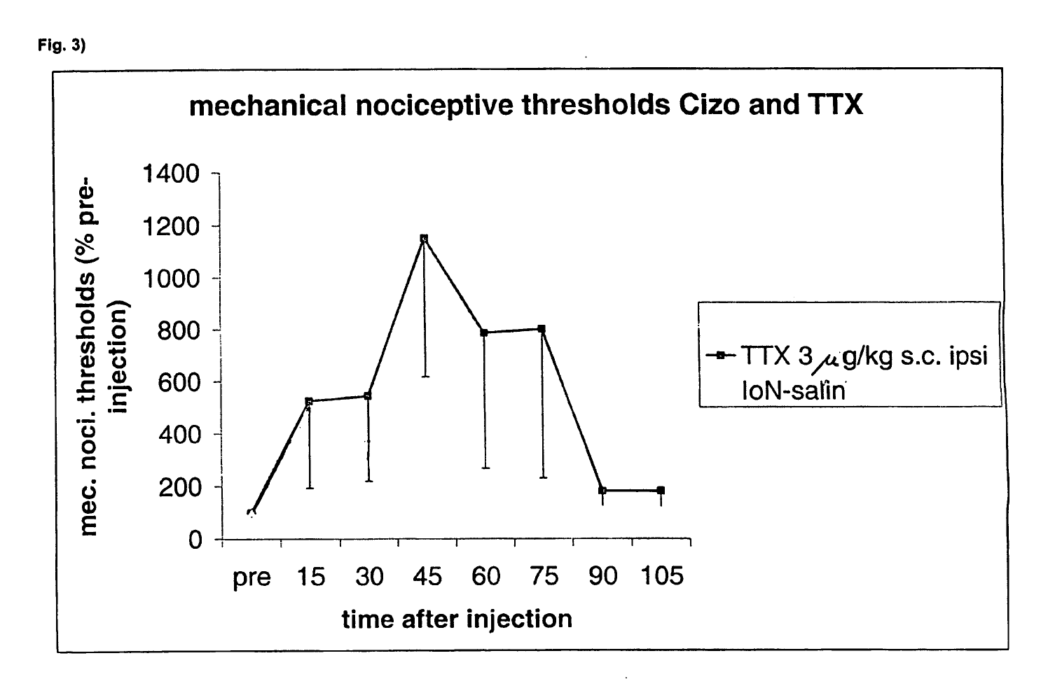

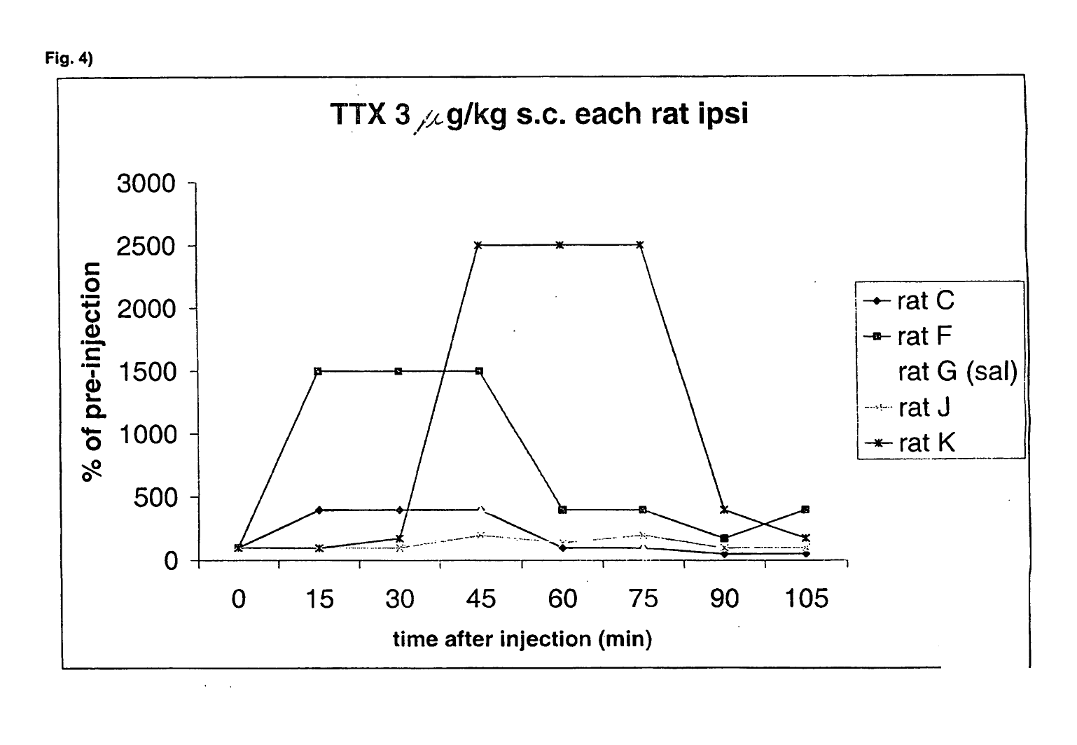

a) with the paw pressure model according to (Randall& Selitto);
vocalization threshold (supra-spinal integrated response); (FIG. 1)
paw withdrawal threshold (spinally-coordinated reflex); (FIG. 2)
   b) with von Frey filaments
threshold response after applying perpendicularly to the plantar surface of hindpaw (FIGS. 3 and 4).

The results are shown in FIGS. 1 to 4.

FIG. 1 shows a clear dose-dependent effect of TTX on vocalization threshold (supra-spinal integrated response) using the paw pressure model according to (Randall& Selitto).

FIG. 2 shows a clear dose-dependent antihyperalgesic effect of TTX on the PWT (paw withdrawal threshold) (spinally-coordinated reflex) using the paw pressure model according to (Randall& Selitto).

FIG. 3 shows a clear and strong (peripheral) effect of TTX on the mechanical nociceptive thresholds, the threshold response after applying perpendicularly to the plantar surface of hindpaw using von Frey filaments.

FIG. 4 is like FIG. 3 just showing each single rat.

1.-17. (canceled)

18. A method for treating peripheral nervous system-derived neuropathic pain comprising administering to a subject presenting peripheral nervous system-derived pain an amount of a sodium channel blocker and/or a derivative, salt or solvate thereof effective to reduce said pain.

19. The method of claim 18, wherein the sodium channel blocker or derivative, salt, or solvate thereof is in the form of a pure stereoisomer or a non-racemic mixture of stereoisomers.

20. The method of claim 18, wherein the sodium channel blocker or derivative is in the form a neutral compound or neutral salt.

21. The method of claim 18, in which the pain presented by the subject is allodynia.

22. The method of claim 18, in which the pain presented by the subject is causalgia.

23. The method of claim 18, in which the pain presented by the subject is hyperalgesia.

24. The method of claim 18, in which the pain presented by the subject is hyperesthesia.

25. The method of claim 18, in which the pain presented by the subject is hyperpathia.

26. The method of claim 18, in which the pain presented by the subject is neuralgia.

27. The method of claim 18, in which the pain presented by the subject is neuritis.

28. The method of claim 18, in which the pain presented by the subject is neuropathy.

29. The method of claim 18, in which tetrodotoxin or a derivative or analog thereof is administered in an amount between 10 μg/day and 4 mg/day.

30. The method of claim 18 in which tetrodotoxin or a derivative or analog thereof that is isolated from a biological source is administered.

31. The method of claim 18 in which synthetic tetrodotoxin or a synthetic derivative or analog thereof is administered.

32. The method of claim 18 in which the sodium channel blocker or derivative or analog thereof is administered parenterally or orally.

33. The method of claim 18, in which the sodium channel blocker or derivative or analog thereof is administered topically.
